(19) United States
(12) Patent Application Publication
(10) Pub. No.: US 2007/0088181 A1
(43) Pub. Date: Apr. 19, 2007

(54) Catalyst for the preparation of ketones from carboxylic acids

(76) Inventors: Alexey V. Ignatchenko, Longview, TX (US); Michelle Manichanh King, Greeley, CO (US); Zhufang Liu, Kingsport, TN (US)

Correspondence Address: BUCHANAN, INGERSOLL & ROONEY PC, POST OFFICE BOX 1404, ALEXANDRIA, VA, 22313-1404, US

(21) Appl. No.: 11/525,105

(22) Filed: Sep. 22, 2006

Related U.S. Application Data
(60) Provisional application No. 60/719,870, filed on Sep. 23, 2005.

(51) Int. Cl. C07C 45/45; B01J 23/00

(52) U.S. Cl. 568/397; 502/309

(57) ABSTRACT

A catalyst for the preparation of ketones from carboxylic acids is described. The catalyst comprises a mixture of zirconium dioxide, titanium dioxide, and one or more metal oxides from Group 1 or 2 of the Periodic Table of Elements. The catalyst can operate at a lower temperature, reduce coke formation, and provide longer lifetime.

### CROSS-REFERENCE TO RELATED APPLICATIONS

This application claims the benefit of the filing date of U.S. Provisional Patent Application No. 60/719,870, filed Sep. 23, 2005; the entire content of which is hereby incorporated by reference.

### FIELD OF THE INVENTION

The present invention relates to a novel catalyst composition containing a mixture of zirconium dioxide, titanium dioxide, and a metal oxide from Group 1 or 2 of the Periodic Table of Elements. The catalyst composition is particularly useful for the preparation of ketones from carboxylic acids. The catalyst composition can operate at a lower temperature, can reduce coke formation, and can have a longer lifetime than a catalyst of zirconia or titania alone.

### BACKGROUND OF THE INVENTION

Ketones are useful organic solvents for painting, agricultural, and other industries. Preparation of ketones from carboxylic acids with metal oxide catalysts at high temperatures has long been known. Catalysts suggested for this process include oxides of thorium, zirconium, titanium, calcium, barium, cerium, chromium, aluminum, lanthanum, neodymium, samarium, etc.

Ketonization reaction requires high energy, so the process normally operates at temperatures above 400° C., often close to 500° C., in order to achieve a high conversion of carboxylic acids. At these temperatures, formation of carbon on the catalyst cannot be avoided. Once carbon is formed, it catalyzes degradation of organic materials to form more carbon. As the result, activity of the catalyst and conversion of carboxylic acids are decreased.

In order to restore conversion of carboxylic acid, the process can be run at a higher temperature. This, however, will generate even more carbon formation. As a result, the catalyst will die and will need to be replaced.

Thus, there's a continuing need in the art to reduce coke formation with these traditional catalysts.

### SUMMARY OF THE INVENTION

One object of the present invention is to obtain a catalyst that operates at low temperature at almost a full conversion of carboxylic acid, so that coke formation is reduced and the catalyst has a longer lifetime. The goal of the present invention was surprisingly achieved by mixing oxides of zirconium and titanium metals.

In one aspect, the present invention relates to a catalyst composition comprising zirconium dioxide, titanium dioxide, and at least one metal oxide from Group 1 or 2 of the Periodic Table of Elements.

In another aspect, the present invention relates to a process for preparing ketones from carboxylic acids. The process comprises the step of contacting at least one carboxylic acid with a catalyst composition comprising zirconium dioxide, titanium dioxide, and at least one metal oxide from Group 1 or 2 of the Periodic Table of Elements at conditions effective to produce a ketone.

### DETAILED DESCRIPTION OF THE INVENTION

The catalyst compositions of the present invention preferably comprise a mixture of zirconium dioxide, titanium dioxide, and one or more metal oxides from Group 1 or 2 of the Periodic Table of Elements.

Mixtures of zirconium dioxide (zirconia) and titanium dioxide (titania) may be prepared by any manner known in the art. Zirconia-titania mixtures are also commercially available such as from Saint-Gobain Norpro. The catalyst can contain varying concentrations of zirconia and titania. Desirable concentrations include 0.1 up to 50 weight percent of titanium dioxide, and 30 to 99.8 weight percent of zirconium dioxide, based on the total weight of the catalyst composition.

The zirconia-titania mixture can be modified with a Group 1 or 2 metal salt which is either basic itself or becomes basic under the reaction conditions. Favorable metals include sodium, potassium, cesium, and lithium from Group 1 and calcium, strontium, barium, and magnesium from Group 2. The other members of Groups 1 and 2 can also produce ketones in the same manner, but they are generally less effective. The more desirable of these promoters include potassium, sodium, rubidium, magnesium, calcium, strontium, and barium. And the most desirable elements include sodium, potassium, and calcium.

Suitable basic counterions of the metal salts include hydroxide, carbonate, and oxide. Counterions that become basic under the reaction conditions either by oxidation or pyrolysis include bicarbonate, carboxylate salts of mono- or poly-basic carboxylic acids containing 1-20 carbon atoms, nitrate, nitrite, or any of various organometallics which under calcining conditions oxidize to hydroxides and oxides.

Incorporating the Group 1 or 2 promoter can take place by several methods. The first is an exchange effected by soaking a solution of the exchanging agent in a suitable solvent with the solid zirconia-titania mixture. The second is by incipient wetness techniques with any amount of exchanging agent. Other methods include co-precipitation of zirconia-titania from a suitable precursor and the promoter simultaneously.

After incorporating the Group 1 or 2 promoter, the catalyst may be dried and/or calcined at elevated temperature. This step is optional since the catalyst will typically be heated in the reactor before it contacts any starting material. During that heating step, the catalyst is effectively dried and/or calcined.

Regardless of the incorporation method, there is a maximum amount of exchanging agent that is optimum. The production of ketones will take place at levels above or below the optimum; however, the production of ketones, especially mixed ketones, will not be optimal at these levels.

The optimum level of catalyst promoter depends on the exact agent. But with an agent such as potassium hydroxide, it will typically fall in the 0.1-20 weight percent range. More desirable levels are found in the 0.25-10 weight percent range. And the most desired loading level is 0.5-5 weight percent, based on the total weight of the catalyst composition.

The catalyst compositions of the present invention are particularly useful in preparing ketones from carboxylic acids. The process comprises the step of contacting at least one carboxylic acid with the catalyst compositions mentioned above at conditions effective to produce a ketone.

Carboxylic acids that can be converted to ketones using the catalysts of the present invention include those having the general formulae (Ia) and (Ib): 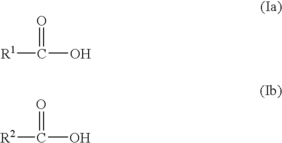
where R¹ and R² may be the same or different and are each independently alkyl, cycloalkyl, arylalkyl, aryl, or hetaryl.

The ketones that are produced have the general formula (II): 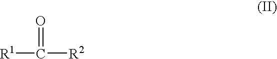
where R¹ and R² are the same as in formulae (Ia) and (Ib). If R¹ is identical to R², then the ketones are symmetrical. If R¹ is not identical to R², then the ketones are asymmetrical.

R¹ and R² are each preferably alkyl having 1 to 17 carbon atoms, cycloalkyl having 3 to 8 ring members, arylalkyl of 7 to 12 carbon atoms, aryl or hetaryl, and one or more of the radicals R¹ and R² carry one or more hydrogen atoms on the α-carbon atom.

Examples of ketones that are obtainable by the process of the present invention from the corresponding acids are diethyl ketone, di-n-propyl ketone, diisopropyl ketone, methyl propyl ketone, methyl isopropyl ketone, ethyl isopropyl ketone, nonan-5-one, octane-2,7-dione, cyclopentanone, cycloheptanone, acetophenone, propiophenone, butyrophenone, isobutyrophenone, valerophenone, phenylacetone, 1,2-diphenylacetone, cyclohexyl methyl ketone, cyclohexyl phenyl ketone, cyclopropyl methyl ketone, pinacolone and even heterocyclic ketones, such as 3-acetylpyridine, 4-acetylpyrazole and 4-acetylimidazole.

The contacting step may be carried out in any reactor known in the art.

The temperatures in the reactive zone preferably are in the 250-550° C. range. More preferably, they exist in the 300-500° C. range. And most preferably, they occur in the 400-450° C. range.

The ketonization reaction can be run over a wide range of pressures. Suitable pressures include 0 to 800 psi. Preferred pressures include 50 to 100 psi.

As used herein, feed rates refer to the quantity of condensed reactants fed through the system regardless of what form they actually exist in the reaction zone. The optimum feed rate varies directly with the temperature, with higher feed rates accompanying the higher temperatures. This feed rate will usually fall in the range of 0.1 to 100 volumes of condensed reactants per volume of catalyst per hour.

The most preferable feed rates are chosen to minimize the amount of unreacted starting materials without pushing the reaction to such extremes that side reactions begin to dominate. As such, the conversion of the least reactive acid is desirably 85-99 percent. A more desirable range is 90-98 percent. And the most desirable conversion of starting acids is 95-97 percent. Although the reaction will take place beyond these limits, below 85 percent conversion will give outstanding overall ketone selectivity with fewer by-products, but will require an additional, more costly distillation during product recovery to separate the unreacted starting material from the product for recycle. And conversions beyond 99 percent begin to entail significant product losses as increasing contributions by side reactions convert already formed product as well as the starting materials into side products.

Proper selection of the ratio of the starting materials can improve the overall success of the reaction. The stoichiometry of mixed ketone preparation suggests a molar ratio of 1:1 of the starting carboxylic acids to achieve the maximum amount of the mixed product while minimizing the production of the two symmetrical ketones. In reality, however, one of the starting acids might be more expendable than the other, so that by using more of the more expendable acid, the yield of asymmetrical ketone product from the less expendable acid increases.

Thus, the choice of which ratio of starting materials to use will depend on the overall objectives, the deviation of the actual catalyst from these statistical limits, and what to do with the by-products.

For this reason, the preferred ratio of starting carboxylic acids is generally in the 5:1 to 1:1 range with the material of less importance being in abundance. A more preferable range to optimize the return without co-producing large amounts of by-product is 3:1 to 1:1. And the most preferable range of starting carboxylic acids is 2:1 to 1:1. In the latter case, the selectivity to the unsymmetrical ketone is good without producing unacceptably large amounts of by-product.

It is possible to feed the carboxylic acids into the reactor with up to 50 weight percent of water. Water can prolong catalyst life by preventing coke formation on the catalyst.

When the activity and/or selectivity of the catalyst decrease due to coking, the promoted zirconia-titania catalyst can be regenerated using a gas containing 0.1-100 percent oxygen at appropriate temperatures for various times, the key being how much carbon dioxide and carbon monoxide exist in the off-gases. A preferable range is 1-20 percent and a more preferable range is 3-10 percent oxygen. Any inert diluent is acceptable including nitrogen, helium, argon, neon, and water. It is possible to use carbon dioxide as the oxidant while monitoring the amount of carbon monoxide existing in the off-gases. In this case, the carbon dioxide serves as both the inert diluent and the source of oxygen. And it may be diluted with any other inert diluent. But using carbon dioxide generally requires higher regeneration temperatures.

Regeneration temperatures generally fall in the 300-700° C. range. More preferably, they exist in the 350-600° C. range. And the most preferable temperatures for catalyst regeneration are 400-500° C. Coincidentally, these are similar temperatures at which the ketonization reaction takes place, albeit in the absence of the regenerating oxidant. At the most preferable regeneration temperatures, the time required to reduce the carbon oxides to 1 percent of their highest level is generally 0.5 to 8 hours with a feed rate of 10 catalyst volumes per hour of the regenerating gas.

This treatment removes up to several weight percent of carbon on the catalyst surface. It also restores essentially complete catalyst activity. The catalyst integrity is unaffected because of the inherent strength of the zirconia-titania material and the fact that the treatment takes place at mild temperatures.

Suitable inert agents to use during the regeneration process include water, nitrogen, carbon dioxide, argon, helium, and neon. The most preferred agents are water and nitrogen solely because they are most readily available and least expensive.

As used herein, the indefinite article “a” means one or more.

Unless otherwise indicated, all numbers expressing quantities of ingredients, properties such as molecular weight, reaction conditions, and so forth used in the description and claims are to be understood as being modified in all instances by the term “about.” Accordingly, unless indicated to the contrary, the numerical parameters set forth in the following description and attached claims are approximations that may vary depending upon the desired properties sought to be obtained by the present invention. At the very least, each numerical parameter should at least be construed in light of the number of reported significant digits and by applying ordinary rounding techniques. Further, the ranges stated in this disclosure and the claims are intended to include the entire range specifically and not just the endpoint(s). For example, a range stated to be 0 to 10 is intended to disclose all whole numbers between 0 and 10 such as, for example 1, 2, 3, 4, etc., all fractional numbers between 0 and 10, for example 1.5, 2.3, 4.57, 6.1113, etc., and the endpoints 0 and 10.

Notwithstanding that the numerical ranges and parameters describing the invention are approximations, the numerical values set forth in the specific examples are reported as precisely as possible. Any numerical value, however, inherently contains certain errors necessarily resulting from the standard deviation found in their respective testing measurements.

The invention is further illustrated and described by the following examples.

### EXAMPLES

Methyl isopropyl ketone (MIPK), which is a useful industrial product and, at the same time, is an example of an unsymmetrical ketone formed by two different carboxylic acids, was chosen as a model compound for Examples 1-3 below. Preparation of an asymmetrical ketone is a good example to demonstrate the effectiveness of the catalyst, due to the different reactivity of the two carboxylic acids. When the more reactive acid is fully converted, the less reactive one still remains in the crude mixture. The less reactive acid requires a higher temperature to complete its conversion. This condition may cause more carbon formation than preparation of a symmetrical ketone.

In the examples below, analyses were completed using Varian 6890+ gas chromatograph equipped with a 30 meter Quadrex 007 CW (carbowax) capillary column, 0.53 mm in diameter, and a thermal conductivity detector.

Yield is defined as the ratio of the number of moles of product obtained to the number of moles of the starting material. Conversion is defined as the ratio of the number of moles of starting material reacted to the number of moles of the starting material. Selectivity is defined as the ratio of the number of moles of product obtained to the number of moles of the starting material reacted.

### Example 1

Catalyst Preparation

100 g of a composition containing 40% by weight titania and 60% by weight zirconia, commercially available from Saint-Gobain Norpro, was soaked in 100 ml of a 10% solution of KOH in water for 24 hrs at 60° C. under vacuum. The KOH solution was drained; the catalyst was washed with 100 ml of deionized water, three times, and dried at 130° C. for 4 hrs.

MIPK Preparation

70 ml of the resulting catalyst were placed in a stainless steel reactor, one inch in diameter. The bottom and the top of the catalyst bed were each filled with 10 ml of glass beads. The reactor was heated inside an electric furnace to a temperature indicated in Table 1 below. A mixture of acetic and isobutyric acid in molar ratio of 1.6:1, having 10% water by weight, was introduced from the top to the bottom of the reactor through a line preheated to 170° C. at the rate of 70 ml/hr. The reaction was conducted at atmospheric pressure. The product was collected every 1-2 hrs in a condenser chilled to 0° C., weighed, and analyzed by GC. This experiment was run three times. The results are summarized in the Table 1.

### Example 2 (Comparative)

Example 1 was repeated, except pure zirconia (−100%), commercially available from Saint-Gobain Norpro, treated with KOH, was used as the catalyst. This experiment was run four times. The results are summarized in Table 1.

### Example 3 (Comparative)

Example 1 was repeated, except pure titania (−100%), in its anatase form, commercially available from Engelhard, treated with KOH, was used as the catalyst. This experiment was run four times. The results are summarized in the Table 1.

TABLE 1
Comparison of catalyst performance for MIPK production.

| | | | | Iso-butyric acid basis | | | | |
|---|---|---|---|---|---|---|---|---|
| Example | | | Temperature, | | MIPK | MIPK | DIPK | DIPK |
| No. | Run | Catalyst | ° C. | Conversion, % | Yield, % | Selectivity, % | Yield, % | Selectivity, % |
| 1 | A | 40% TiO₂/60% ZrO₂ + KOH | 450 | 98.0 | 70.8 | 72.1 | 21.3 | 21.7 |
| 1 | B | 40% TiO₂/60% ZrO₂ + KOH | 450 | 98.4 | 62.5 | 63.4 | 20.4 | 20.7 |
| 1 | C | 40% TiO₂/60% ZrO₂ + KOH | 450 | 98.1 | 70.2 | 71.4 | 21.5 | 21.9 |
| 2 | D | ZrO₂+ KOH | 475 | 94.1 | 62.7 | 66.3 | 18.5 | 9.8 |
| 2 | E | ZrO₂ + KOH | 475 | 95.1 | 63.0 | 65.9 | 22.4 | 11.8 |
| 2 | F | ZrO₂ + KOH | 475 | 95.7 | 63.1 | 65.7 | 16.6 | 8.6 |
| 2 | G | ZrO₂ + KOH | 475 | 95.1 | 67.5 | 70.7 | 19.7 | 10.3 |
| 3 | H | TiO₂ + KOH | 475 | 99.5 | 62.0 | 62.2 | 20.7 | 22.6 |
| 3 | I | TiO₂ + KOH | 475 | 99.8 | 66.5 | 66.7 | 19.7 | 20.5 |
| 3 | J | TiO₂ + KOH | 475 | 94.4 | 67.2 | 70.9 | 13.2 | 14.9 |
| 3 | K | TiO₂ + KOH | 475 | 93.4 | 65.4 | 69.7 | 12.0 | 14.3 |

| | | | | | | | GC | |
|---|---|---|---|---|---|---|---|---|
| | Acetic acid basis | | | | | | Accountability | |
| Example | | MIPK | MIPK | Acetone | Acetone | Mass | Organic | Aqueous |
| No. | Conversion, % | Yield, % | Selectivity, % | Yield, % | Selectivity, % | Balance, % | Phase % | Phase % |
| 1 | 99.8 | 44.2 | 44.3 | 45.5 | 45.6 | 96.2 | 97.2 | 99.9 |
| 1 | 99.9 | 39.0 | 39.1 | 41.0 | 41.0 | 88.4 | 96.7 | 99.7 |
| 1 | 99.9 | 43.8 | 43.9 | 47.0 | 47.0 | 96.5 | 96.7 | 99.7 |
| 2 | 99.6 | 39.2 | 39.3 | 43.8 | 22.0 | 88.9 | 97.7 | 100.1 |
| 2 | 99.7 | 39.3 | 39.4 | 45.4 | 22.8 | 89.5 | 99.3 | 100.0 |
| 2 | 99.7 | 39.4 | 39.5 | 59.1 | 29.6 | 93.8 | 99.4 | 100.0 |
| 2 | 99.7 | 42.2 | 42.3 | 52.8 | 26.5 | 95.9 | 99.2 | 100.0 |
| 3 | 100.0 | 38.7 | 38.7 | 30.5 | 15.2 | 86.1 | 96.4 | 99.3 |
| 3 | 100.0 | 41.5 | 41.5 | 34.3 | 17.2 | 89.1 | 96.9 | 99.2 |
| 3 | 99.4 | 42.0 | 42.2 | 31.5 | 15.8 | 89.2 | 97.4 | 99.9 |
| 3 | 99.3 | 40.9 | 41.2 | 30.3 | 15.2 | 86.3 | 97.1 | 100.0 |

As seen in Table 1, at temperature 475° C., both pure titania and zirconia catalysts gave approximately the same yield of MIPK. However, conversion of isobutyric acid with pure zirconia catalyst was lower than with pure titania catalyst under exactly the same conditions.

It was unexpected and surprising that the mixture of titania and zirconia was a better catalyst than any of these metal oxides in their pure form. Conversion of isobutyric acid with the new catalyst has improved compared to the zirconia catalyst, and it was achieved at a lower temperature, than with the titania catalyst. Because the mixture of titania and zirconia can achieve about the same conversion at a lower temperature than the titania catalyst, less coke formation and longer catalyst lifetime can be expected for the catalyst mixture. At the same time, the yield and the selectivity were comparable or slightly higher with the mixed catalyst than with any of the metal oxides in their pure form.

### Example 4

Pinacolone Preparation

Example 1 was repeated, except a mixture of acetic and pivalic (trimethylacetic) acid in molar ratios of 1:1 and 3:1, having 10% water by weight, was introduced from the top to the bottom of the reactor through a line preheated to 170° C. at the rate of 70-140 ml/hr. The reaction was conducted at atmospheric pressure. The product was collected every 1-2 hrs in a condenser chilled to 0° C., weighed, and analyzed by GC. This experiment was run eight times at different temperatures. The results are summarized in the Table 2.

### Example 5 (Comparative)

Example 4 was repeated, except pure zirconia (−100%), commercially available from Saint-Gobain Norpro, treated with KOH, was used as the catalyst. This experiment was run six times. The results are summarized in Table 2.

### Example 6 (Comparative)

Experiment 4 was repeated, except pure titania (˜100%), in its anatase form, commercially available from Engelhard, treated with KOH, was used as the catalyst. This experiment was run three times. The results are summarized in the Table 2.

TABLE 2
Comparison of catalyst performance for pinacolone production.

| | | | | | | Pivalic acid basis | | |
|---|---|---|---|---|---|---|---|---|
| | | | Acid molar | | LSTV | | Pinacolone | Pinacolone |
| Example No. | Run | Catalyst | ratio A:P | Temp. (° C.) | (hr-1) | Conversion, % | Yield, % | Selectivity, % |
| 4 | A | 40% TiO₂/60% ZrO₂ + KOH | 1.0 | 475 | 2 | 30.2 | 19.9 | 65.8 |
| 4 | B | 40% TiO₂/60% ZrO₂ + KOH | 1.0 | 475 | 1 | 47.8 | 27.6 | 57.8 |
| 4 | C | 40% TiO₂/60% ZrO₂ + KOH | 1.0 | 500 | 1 | 68.9 | 32.2 | 46.7 |
| 4 | D | 40% TiO₂/60% ZrO₂ + KOH | 1.0 | 500 | 1 | 67.1 | 38.2 | 56.9 |
| 4 | E | 40% TiO₂/60% ZrO₂ + KOH | 1.0 | 525 | 1 | 90.1 | 40.2 | 44.6 |
| 4 | F | 40% TiO₂/60% ZrO₂ + KOH | 3.0 | 475 | 2 | 38.8 | 36.8 | 94.8 |
| 4 | G | 40% TiO₂/60% ZrO₂ + KOH | 3.0 | 500 | 1 | 89.5 | 73.1 | 81.7 |
| 4 | H | 40% TiO₂/60% ZrO₂ + KOH | 3.0 | 525 | 1 | 98.1 | 83.1 | 84.7 |
| 5 | I | ZrO₂ + KOH | 1.0 | 475 | 1 | 33.3 | 33.2 | 99.4 |
| 5 | J | ZrO₂ + KOH | 1.0 | 500 | 1 | 41.3 | 39.2 | 94.8 |
| 5 | K | ZrO₂ + KOH | 1.0 | 525 | 1 | 61.5 | 48.7 | 79.1 |
| 5 | L | ZrO₂ + KOH | 3.0 | 475 | 1 | 67.7 | 68.8 | 101.6 |
| 5 | M | ZrO₂ + KOH | 3.0 | 500 | 1 | 94.0 | 90.8 | 96.6 |
| 5 | N | ZrO₂ + KOH | 3.0 | 525 | 1 | 98.7 | 89.3 | 90.5 |
| 6 | O | TiO₂ + KOH | 3.0 | 475 | 1 | 63.6 | 58.5 | 91.9 |
| 6 | P | TiO₂ + KOH | 3.0 | 500 | 1 | 71.0 | 60.4 | 85.0 |
| 6 | Q | TiO₂ + KOH | 3.0 | 525 | 1 | 76.3 | 60.1 | 78.7 |

| | Acetic acid basis | | | | | | GC Accountability | |
|---|---|---|---|---|---|---|---|---|
| | | Pinacolone | Pinacolone | Acetone | Acetone | Mass | Organic | Aqueous |
| Example No. | Conversion, % | Yield, % | Selectivity, % | Yield, % | Selectivity, % | Balance, % | Phase % | Phase % |
| 4 | 99.8 | 19.9 | 19.9 | 85.4 | 85.6 | 97.6 | 98.1 | 99.6 |
| 4 | 100.0 | 27.6 | 27.6 | 67.8 | 67.8 | 91.1 | 96.2 | 99.8 |
| 4 | 100.0 | 32.2 | 32.2 | 57.8 | 57.8 | 80.0 | 96.6 | 100.0 |
| 4 | 100.0 | 38.2 | 38.2 | 53.0 | 53.0 | 83.5 | 95.7 | 99.7 |
| 4 | 100.0 | 40.2 | 40.2 | 39.9 | 39.9 | 68.7 | 92.8 | 98.8 |
| 4 | 99.8 | 12.3 | 12.3 | 86.5 | 86.8 | 100.2 | 99.3 | 100.0 |
| 4 | 100.0 | 24.4 | 24.4 | 61.9 | 61.9 | 92.7 | 96.5 | 100.0 |
| 4 | 100.0 | 27.7 | 27.7 | 57.6 | 57.6 | 91.0 | 95.9 | 99.5 |
| 5 | 100.0 | 33.2 | 33.2 | 69.8 | 69.8 | 100.3 | 99.3 | 99.3 |
| 5 | 100.0 | 39.2 | 39.2 | 63.1 | 63.1 | 99.9 | 96.7 | 99.5 |
| 5 | 100.0 | 48.7 | 48.7 | 49.3 | 49.3 | 96.5 | 95.1 | 99.3 |
| 5 | 100.0 | 22.9 | 22.9 | 72.2 | 72.2 | 99.8 | 99.3 | 100.0 |
| 5 | 99.9 | 30.3 | 30.3 | 58.5 | 58.5 | 95.3 | 96.5 | 99.6 |
| 5 | 99.9 | 29.8 | 29.8 | 48.2 | 48.2 | 88.0 | 94.1 | 99.8 |
| 6 | 99.1 | 19.5 | 19.7 | 75.3 | 76.0 | 98.4 | 97.3 | 100.0 |
| 6 | 99.5 | 20.1 | 20.2 | 68.2 | 68.5 | 95.6 | 96.0 | 100.0 |
| 6 | 99.0 | 20.0 | 20.2 | 70.7 | 71.4 | 94.0 | 96.9 | 99.5 |

As seen from Table 2, a higher conversion of pivalic acid and a higher pinacolone yield were achieved with the mixed titania-zirconia catalyst than with the pure titania catalyst (83% yield in run H vs. 60% yield in run Q). The results were even slightly better with the pure zirconia catalyst (89% yield in run N). However, the increment of improvement does not justify the increase in the catalyst cost. Zirconia is more expensive than titania, so increasing zirconia content above 60% is not necessary.

### Example 7

Methyl Ethyl Ketone (MEK) Preparation

Example 1 was repeated, except a mixture of acetic and propionic acid in molar ratio of 1:1, having 10% water by weight, was introduced from the top to the bottom of the reactor through a line preheated to 170° C. at the rate of 70 ml/hr. The reaction was conducted at atmospheric pressure. The product was collected every 1-2 hrs in a condenser chilled to 0° C., weighed, and analyzed by GC. This experiment was run two times at different temperatures. The results are summarized in the Table 3.

### Example 8 (Comparative)

Example 7 was repeated, except pure zirconia (˜100%), commercially available from Saint-Gobain Norpro, treated with KOH, was used as the catalyst. This experiment was run two times. The results are summarized in Table 3.

### Example 9 (Comparative)

Experiment 7 was repeated, except pure titania (˜100%), in its anatase form, commercially available from Engelhard, treated with KOH, was used as the catalyst. This experiment was run three times. The results are summarized in the Table 3.

TABLE 3
Comparison of catalyst performance for MEK production.

| | | | | | Propionic acid basis | | |
|---|---|---|---|---|---|---|---|
| | | | | Bottom Temp. | | | MEK |
| Example No. | Run | Catalyst | Top Temp. (° C.) | (° C.) | Conversion, % | MEK Yield, % | Selectivity, % |
| 7 | R | 40% TiO₂/60% ZrO₂ + KOH | 345 | 400 | 99.7 | 47.7 | 47.8 |
| 7 | S | 40% TiO₂/60% ZrO₂ + KOH | 400 | 450 | 100 | 45.9 | 45.9 |
| 8 | T | ZrO₂ + KOH | 350 | 400 | 99.8 | 49.0 | 49.2 |
| 8 | U | ZrO₂ + KOH | 410 | 450 | 100 | 48.0 | 48.0 |
| 9 | V | TiO₂ + KOH | 370 | 400 | 87.6 | 45.1 | 51.4 |
| 9 | W | TiO₂ + KOH | 420 | 450 | 97.6 | 47.0 | 48.2 |

| | Acetic acid basis | | | | | | GC Accountability | |
|---|---|---|---|---|---|---|---|---|
| | | | MEK | Acetone | Acetone | Mass | Organic | Aqueous |
| Example No. | Conversion, % | MEK Yield, % | Selectivity, % | Yield, % | Selectivity, % | Balance % | Phase % | Phase % |
| 7 | 99.9 | 47.7 | 38.8 | 38.8 | 91.5 | 98.6 | 100 | 47.7 |
| 7 | 100 | 45.9 | 37.4 | 37.4 | 90.3 | 97.0 | 99.6 | 45.9 |
| 8 | 99.9 | 49.1 | 39.6 | 39.6 | 93.3 | 98.8 | 100 | 49.0 |
| 8 | 100 | 48.0 | 39.6 | 39.6 | 93.7 | 97.5 | 99.6 | 48.0 |
| 9 | 90.2 | 49.9 | 35.4 | 39.3 | 92.8 | 99.6 | 100 | 45.1 |
| 9 | 98.5 | 47.7 | 35.9 | 36.4 | 90.8 | 97.3 | 100 | 47.0 |

Comparable acid conversion and ketone yield were achieved with mixed titania-zirconia catalysts and zirconia catalysts. The results with the zirconia catalysts were better than with the titania catalyst. The lowest temperature on the top of the catalyst bed was observed for the mixed titania-zirconia catalyst (run R). Thus, this catalyst showed the potential of making less coke by running at a lower temperature, which is one of the objects of the current invention.

### Example 10

Methyl Isobutyl Ketone (MIBK) Preparation

Example 1 was repeated, except a mixture of acetic and isovaleric acid in molar ratios of 1:1 and 2:1, having 10% water by weight, was introduced from the top to the bottom of the reactor through a line preheated to 170° C. at the rate of 70 ml/hr. The reaction was conducted at atmospheric pressure. The product was collected every 1-2 hrs in a condenser chilled to 0° C., weighed, and analyzed by GC. This experiment was run four times at different temperatures. The results are summarized in the Table 4.

### Example 11 (Comparative)

Example 10 was repeated, except pure zirconia (−100%), commercially available from Saint-Gobain Norpro, treated with KOH, was used as the catalyst. This experiment was run four times. The results are summarized in Table 4.

### Example 12 (Comparative)

Experiment 10 was repeated, except pure titania (−100%), in its anatase form, commercially available from Engelhard, treated with KOH, was used as the catalyst. This experiment was run four times. The results are summarized in the Table 4.

TABLE 4
Comparison of catalyst performance for MIBK Production.

| | | | | | | Isovaleric acid basis | | |
|---|---|---|---|---|---|---|---|---|
| Example | | | Acid molar | Top Temp. | Bottom Temp. | Conversion, | MIBK | MIBK |
| No | Run | Catalyst | ratio A:P | (° C.) | (° C.) | % | Yield, % | Selectivity, % |
| 10 | X | 40% TiO₂/60% ZrO₂ + KOH | 1 | 400 | 450 | 87.6 | 31.5 | 35.9 |
| 10 | Y | 40% TiO₂/60% ZrO₂ + KOH | 1 | 420 | 475 | 98.8 | 34.7 | 35.2 |
| 11 | Z | ZrO₂ + KOH | 1 | 410 | 450 | 96.8 | 40.8 | 42.2 |
| 11 | AA | ZrO₂ + KOH | 1 | 440 | 475 | 99.9 | 41.5 | 41.5 |
| 12 | AB | TiO₂ + KOH | 1 | 410 | 450 | 78.7 | 44.2 | 56.1 |
| 12 | AC | TiO₂ + KOH | 1 | 440 | 475 | 90.7 | 42.7 | 47.1 |
| 10 | AD | 40% TiO₂/60% ZrO₂ + KOH | 2 | 400 | 450 | 95.0 | 47.0 | 49.4 |
| 10 | AE | 40% TiO₂/60% ZrO₂ + KOH | 2 | 420 | 475 | 99.8 | 58.2 | 58.3 |
| 11 | AF | ZrO₂ + KOH | 2 | 405 | 450 | 99.4 | 60.8 | 61.2 |
| 11 | AG | ZrO₂ + KOH | 2 | 430 | 475 | 99.9 | 63.1 | 63.2 |
| 12 | AH | TiO₂ + KOH | 2 | 410 | 450 | 90.1 | 60.7 | 67.4 |
| 12 | AI | TiO₂ + KOH | 2 | 450 | 475 | 95.2 | 58.5 | 61.5 |

| | Acetic acid basis | | | | | | GC Accountability | |
|---|---|---|---|---|---|---|---|---|
| Example | | MIBK | MIBK | Acetone | Acetone | Mass | Organic | Aqueous |
| No | Conversion, % | Yield, % | Selectivity, % | Yield, % | Selectivity, % | Balance, % | Phase % | Phase % |
| 10 | 100.0 | 31.5 | 31.5 | 53.7 | 53.7 | 100.0 | 98.1 | 99.8 |
| 10 | 100.0 | 34.7 | 34.7 | 42.5 | 42.5 | 96.1 | 99.2 | 99.9 |
| 11 | 100.0 | 40.9 | 40.9 | 42.0 | 42.0 | 100.8 | 99.4 | 99.8 |
| 11 | 99.9 | 41.5 | 41.5 | 36.1 | 36.1 | 100.3 | 98.3 | 100 |
| 12 | 97.6 | 44.2 | 45.3 | 46.0 | 47.1 | 101.1 | 99.1 | 99.8 |
| 12 | 98.8 | 42.8 | 43.3 | 42.9 | 43.4 | 101.2 | 98.8 | 99.7 |
| 10 | 99.9 | 23.4 | 23.5 | 57.9 | 57.9 | 95.2 | 98.5 | 99.6 |
| 10 | 99.9 | 29.0 | 29.1 | 55.7 | 55.7 | 101.1 | 97.7 | 99.8 |
| 11 | 99.8 | 30.4 | 30.4 | 53.5 | 53.6 | 99.9 | 98.5 | 99.7 |
| 11 | 99.9 | 31.5 | 31.6 | 50.5 | 50.6 | 96.1 | 97.7 | 99.7 |
| 12 | 98.5 | 30.3 | 30.8 | 56.6 | 57.4 | 96.4 | 99.0 | 99.6 |
| 12 | 99.7 | 29.2 | 29.3 | 51.9 | 52.1 | 91.0 | 98.0 | 99.7 |

Comparable MIBK yield was achieved with mixed titania-zirconia catalysts and titania catalysts (see runs AE and AI in the Table 4). However, the mixed titania-zirconia catalyst achieved that comparable yield at a temperature 30 degrees lower than the titania catalyst. In general, the new catalyst provided 10-30 degrees lower temperature than pure zirconia or titania in other runs. Pure zirconia could probably run even at lower temperatures to achieve the same degree of conversion and yield. However, a high price of zirconia makes our catalyst economically more attractive. Thus, the new catalyst can be used at a lower temperature so that it could make less coke and have a longer lifetime.

The invention has been described in detail with particular reference to preferred embodiments thereof, but it will be understood that variations and modifications can be effected within the spirit and scope of the invention.

We claim:

1. A catalyst composition comprising zirconium dioxide, titanium dioxide, and at least one metal oxide from Group 1 or 2 of the Periodic Table of Elements.

2. The catalyst composition according to claim 1, wherein the metal in said metal oxide is lithium, sodium, potassium, cesium, calcium, strontium, barium, or magnesium.

3. The catalyst composition according to claim 1, wherein the metal in said metal oxide is sodium, potassium, or calcium.

4. A catalyst composition comprising 0.1 to 20 weight percent of a metal oxide from Group 1 or 2 of the Periodic Table of Elements, 0.1 to 50 weight percent of titanium dioxide, and 30 to 99.8 weight percent of zirconium dioxide.

5. The catalyst composition according to claim 4, which comprises 0.25 to 10 weight percent of said metal oxide.

6. The catalyst composition according to claim 4, which comprises 0.5 to 5 weight percent of said metal oxide.

7. The catalyst composition according to claim 4, wherein the metal in said metal oxide is sodium, potassium, or calcium.

8. A process for preparing ketones from carboxylic acids, said process comprising the step of contacting at least one carboxylic acid with a catalyst composition comprising zirconium dioxide, titanium dioxide, and at least one metal oxide from Group 1 or 2 of the Periodic Table of Elements at conditions effective to produce a ketone.

9. The process according to claim 8, wherein the metal in said metal oxide is lithium, sodium, potassium, cesium, calcium, strontium, barium, or magnesium.

10. The process according to claim 8, wherein the metal in said metal oxide is sodium, potassium, or calcium.

11. The process according to claim 8, wherein said catalyst composition comprises 0.1 to 20 weight percent of the metal oxide, 0.1 to 50 weight percent of titanium dioxide, and 30 to 99.8 weight percent of zirconium dioxide.

12. The process according to claim 8, wherein said catalyst composition comprises 0.25 to 10 weight percent of said metal oxide.

13. The process according to claim 8, wherein said catalyst composition comprises 0.5 to 5 weight percent of said metal oxide.

14. The process according to claim 8, wherein said carboxylic acid is acetic acid and isobutyric acid, and said ketone is methyl isopropyl ketone.

15. A process for preparing methyl isopropyl ketone, said process comprising the step of contacting acetic acid and isobutyric acid with a catalyst composition comprising zirconium dioxide, titanium dioxide, and a metal oxide of sodium, potassium, or calcium at conditions effective to form methyl isopropyl ketone.
